(19) United States
(12) Patent Application Publication
(10) Pub. No.: US 2006/0084925 A1
(43) Pub. Date: Apr. 20, 2006

(54) Medical syringe with colored plunger and transparent barrel assembly

(76) Inventor: J.W. Max Ramsahoye, Wainwright (CA)

Correspondence Address: GENE SCOTT; PATENT LAW & VENTURE GROUP, 3140 RED HILL AVENUE, SUITE 150, COSTA MESA, CA, 92626-3440, US

(21) Appl. No.: 10/969,593

(22) Filed: Oct. 20, 2004

(51) Int. Cl. A61M 5/00; A61M 5/315

(52) U.S. Cl. 604/189; 604/218

(57) ABSTRACT

A plurality of medical syringes, each of the syringes having a barrel engaging a plunger and a cannula. The plunger is movable in translation within the barrel and the cannula is fixed at one end of the barrel, and is in fluid communication with the barrel. The barrel is made of a transparent material and has graduation indicia on its side wall. The plunger is of a non-transparent color contrasting with the graduation indicia whereby, the indicia is easily seen against the contrasting background of the plunger. The syringes each have a unique plunger color whereby, each of the medical syringes is able to be selected by color and associated with a particular medication or use.

### RELATED APPLICATIONS

none

### INCORPORATION BY REFERENCE

Applicant hereby incorporates herein by reference, the U.S. patents and U.S. patent applications referred to in the Description of Related Art section of this application, as filed.

### BACKGROUND OF THE INVENTION

1. Field of the Invention

This invention relates generally to medical syringes and more particularly to such a syringe with colored plunger. The invention further relates to such colored syringes with color coding related to use.

2. Description of Related Art

The following art defines the present state of this field and each disclosure is hereby incorporated herein by reference:

Hunt, U.S. Pat. No. 2,888,015 describes a hypodermic syringe including a barrel of light transmitting material having at one end an inlet for the attachment of a conventional hollow hypodermic needle and finger gripping portion adjacent to its opposite end, said barrel having a plurality of areas of differing colors, extending from adjacent the inlet end toward the opposite end, said areas of differing colors each extending the same length axially of the barrel and providing an indicia of the volume of fluid contained within the barrel, and a piston slideable within said barrel.

Howe, U.S. Pat. No. 5,242,405 describes a syringe barrel having graphics visualization features that includes an elongate body portion having a longitudinal axis and a sidewall spaced from the axis defining an inside diameter and a chamber for retaining fluid. The sidewall includes a transparent portion having indicia and contrast means of substantially uniform color opposed from the indicia for improving readability of the indicia.

Ryan, U.S. Pat. No. 5,292,308 describes four safety line connectors that include a single piece male connector and a two-piece female connector. The male connector has a luer on one end, one or more inwardly extending bayonet recesses on a middle portion and a reduced diameter second end enclosing a resilient septum. The female connector has a luer on one end and a two part receiving cylinder on the other with inwardly extending knobs. The luer and the first part of the cylinder are separated by a wall through which a hollow needle extends. The connectors are mated by sliding the second end of the male connector into the receiving cylinder of the female connector with the knobs of the female connector being guided by the recesses of the male connector. The connectors are locked into place by bringing them together, and then rotating them such that knobs move past restrictions in the recesses and lock into place. The parts of the female connector are joined together by sonic welding or solvent bonding which is enhanced by providing one with a reduced inner diameter end and the other with a reduced outer diameter end so that the parts join in a male/female relationship. The ends of the two parts are preferably faceted to prevent relative rotation of the joined parts. The second part of the cylinder is advantageously color coded to identify the type or needle being carried.

Demark, U.S. Pat. No. 5,328,466 describes a syringe and needle assembly having a means to align the beveled tip of the needle with the volumetric scale on the syringe. In the preferred embodiment, a visible mark is placed longitudinally on the hub of the needle on the same side and in coaxial alignment with the beveled tip of the needle. This allows for the needle to be installed on the syringe and the beveled tip quickly aligned with the volumetric scale printed on the syringe body without the need for removing the needle safety cap prior to or during installation.

Souryal, U.S. Pat. No. 5,468,224 describes a method where injectable medications are color coded by adding harmless coloring materials to the medications themselves. In one embodiment of the invention, each medication is assigned a color. In another embodiment, a class of medications is assigned a color.

Caulfield et al., U.S. Pat. No. 5,692,640 describes a system for establishing and maintaining the identity of a medication in a syringe from the point in time that a medication is withdrawn from a vial to the point in time that the medication is administered to a patient. This method is based on integrating a label dispenser including preprinted, pressure sensitive, syringe labels with a medicine vial. As the medication is withdrawn from the vial a label is provided that identifies the contents of the vial. When this label is applied to the syringe it provides the ability to verify that the correct medication and dosage is being administered to the correct patient. Subsequently the label can be removed from the syringe and used to provide a permanent record of the administration.

Broselow, U.S. Pat. No. 6,132,416 describes a method of determining the proper medication dosage for a patient, including the steps of correlating a plurality of dosages of a plurality of medications to respective weight-related values indicative of the proper dosage of each of the medications for a given patient weight range, assigning a color to each of the respective weight-related values within a predetermined, fixed sequence of colors collectively representing a dosage range for each of the medications, wherein each of the colors represents a proper dosage for each medication for a patient having a weight within the weight range to which the color is assigned, and providing a dosing system which correlates each of the dosages of the medications with the assigned color within the fixed sequence of colors without regard to the potency of said medication.

Aydelotte et al., U.S. Pat. No. 6,290,678 describes a syringe having color demarcation features to enhance readability. Said syringe having a central body portion that is a barrel having a proximal and distal end along a central longitudinal axis that acts as a reservoir for fluid inside the syringe. The sidewall of the barrel is relatively transparent having volume demarcations in contrasting colors to increase the readability of the demarcations. A plunger and coupled seal member can be engaged with the proximal end of the barrel creating a liquid tight reservoir inside the syringe. The seal member can be made a contrasting color to further enhance the readability of the demarcations.

Our prior art search with abstracts described above teaches: a high contrast syringe, a color divided hypodermic syringe, a syringe having graphics visualization features, a three-piece intravenous line connector, a syringe and needle assembly, methods of color coding injectable medications, a syringe content identification system, and a universal medication dosing system. Thus, the prior art shows that is it is desirable to color code the apparatus for injecting fluids for safety and related reasons. However, the prior art fails to teach the use of color coded plunger assemblies for providing improved readability to the marks on the barrel of a syringe, and for assuring that the fluid injected is that which is desired by relating various colors to associated fluids in a coding system. The present invention fulfills these needs and provides further related advantages as described in the following summary.

### SUMMARY OF THE INVENTION

The present invention teaches certain benefits in construction and use which give rise to the objectives described below.

A hypodermic syringe consists of a cylindrical barrel having a distal end designed to be connected to a needle or other fluid delivery device and a plunger rod having a stopper at its distal end. The stopper creates a fluid-tight seal between the inside of the syringe barrel and the plunger rod. Axial displacement of the plunger moves the stopper along the inside of the barrel and causes liquid to either be withdrawn or expelled at the syringe's distal opening. Movement of the plunger and stopper creates a cavity of variable volume that can hold liquids such as medications. The overall length of the plunger rod is such that it can always be accessed to adjust the volume of the cavity and therefore withdraw or expel liquids. Since the plunger rod is always accessible it is also always visible on the syringe. The syringe barrel is usually constructed of rigid materials such as thermoplastic and glass. The plunger rod is also rigid and can be constructed out of similar materials. The stopper can be made out of natural rubber, synthetic rubber, or thermoplastic elastomers. Syringe barrels usually contain volume indicia markings on them to indicate the volume of the chamber produced by movement of the plunger rod. The volume demarcations are most usually black in color. The problem of mistakenly administering one medication via a syringe that is pre-drawn (having a volume of fluid within its chamber) when another was intended is readily possible when more than one syringe is present, as for instance, on a medical tray. This error has been coined ‘syringe swap.’ Medication administration by syringes is usually via intravenous (IV) or intramuscular (IM) injection. IV and IM administered drugs often have a rapid-onset of action and therefore erroneous administration can quickly produce negative consequences. Since a vast majority of injectible medications are colorless it may often be impossible to distinguish one type of medication from another when drawn up in same size syringes and therefore the possibility of confusion is real. Certain fields of current medical practice often entail the delivery of several medications at one time or in close chronological proximity. Medications are often drawn into syringes prior to delivery. The order of medication administration is important and may be critical. Current practice in administration of anesthesia is one area where multiple medications are administered over a specific time period and where medication order is important. Color-coded labels are commonly used but these are usually small in size and not visible from all angles. Such labels have been known to detach from their syringe due to faulty adhesive or attachment method. It is desirable to have syringes that are easily identifiable and distinguishable from each other without the possibility of mistake. A standard color coding scheme might be used, as for example, blue for narcotics, orange for paralytics, and violet for vasoactive drugs, however, currently, no universally accepted color-coding system is used. It is desirable to provide a system wherein color is used as an indicator of type of medication or order of administration, where the color does not interfere with viewing visible characteristics of the fluid held in a syringe, including its color, and where the color is useful in reading graduations on a syringe barrel. The present invention provides these advantages.

In one of the best mode preferred embodiments of the present invention, a plurality of medical syringes, each have a barrel engaging a plunger and a cannula. The plunger is movable in translation within the barrel and the cannula is fixed at one end of the barrel, and is in fluid communication with the barrel. The barrel is made of a transparent material and has graduation indicia on its side wall. The plunger is of a non-transparent color contrasting with the graduation indicia whereby, the indicia is easily seen against the contrasting background of the plunger. The syringes each have a unique plunger color whereby, each of the medical syringes is able to be selected by color and associated with a particular medication or order of use.

A primary objective of the present invention is to provide an apparatus and method of use of such apparatus that yields advantages not taught by the prior art.

Another objective of the invention is to enable a distinguishing feature between otherwise identical syringes.

A further objective of the invention is to provide such a feature which does not interfere with viewing a fluid within the syringe or which might detach from the syringe.

A still further objective of the invention is to provide a basis for a color coding scheme for providing a safe use environment when plural syringes are used.

Other features and advantages of the embodiments of the present invention will become apparent from the following more detailed description, taken in conjunction with the accompanying drawings, which illustrate, by way of example, the principles of at least one of the possible embodiments of the invention.

### BRIEF DESCRIPTION OF THE DRAWINGS

The accompanying drawings illustrate at least one of the best mode embodiments of the present invention. In such drawings:

FIG. 1 is a perspective view of a typical medical syringe showing a plunger in position for insertion into a syringe barrel;

FIG. 2 shows the syringe of FIG. 1 with the plunger partially inserted into the barrel;

FIG. 3 shows the syringe of FIG. 1 with the plunger fully inserted into the barrel; and

FIG. 4 is a perspective view of four such syringes showing the appearance when colored plungers are used.

### DETAILED DESCRIPTION OF THE INVENTION

The above described drawing figures illustrate the present invention in at least one of its preferred, best mode embodiments, which is further defined in detail in the following description. Those having ordinary skill in the art may be able to make alterations and modifications in the present invention without departing from its spirit and scope. Therefore, it must be understood that the illustrated embodiments have been set forth only for the purposes of example and that they should not be taken as limiting the invention as defined in the following.

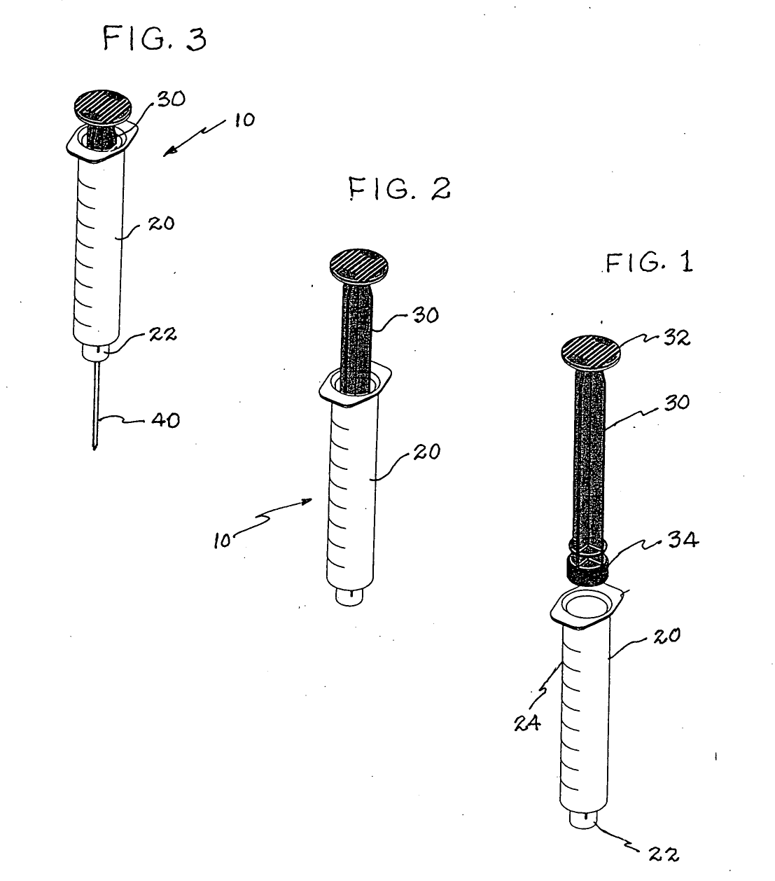

In one embodiment of the present invention a medical syringe 10, as shown in FIGS. 1-3, comprises a barrel 20 engaging a plunger 30 which has a proximal flange 32, and a cannula or needle 40. The plunger 30 is movable in translation within the barrel 20 as manipulated by the flange 32, while the cannula 40 (FIG. 3) is fixed at one end 22 of the barrel 20, and is in fluid communication with the barrel 20. Such a medical syringe 10 is in common use in the medical industry. The barrel 20 is made of a transparent material, preferably glass or plastic, and supports graduation indicia 24 on its side wall as is also well known. The plunger 30 is preferably made of a non-transparent material with a color contrasting with the graduation indicia 24 whereby, the indicia 24 is easily seen against the contrasting background of the plunger 30.

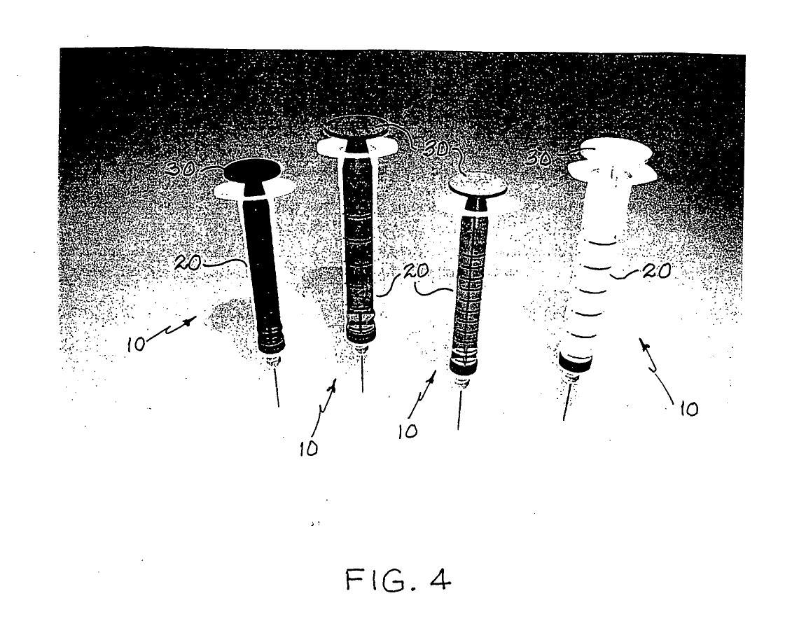

In another embodiment of the present invention, a plurality of the above described medical syringes 10 are provided, as shown in FIG. 4, wherein each of the syringes 10 is of the type described and each has a plunger 30 of a unique color relative to the others. comprising: a barrel engaging a plunger and a cannula, the plunger movable in translation within the barrel, the cannula fixed at one end of the barrel and in fluid communication with the barrel; the barrel of a transparent material and supporting graduation indicia on a side wall of the barrel; the plunger of a non-transparent color contrasting with the graduation indicia whereby, the indicia is easily seen against the contrasting background of the plunger; the plurality of medical syringes each having a unique plunger color whereby, each of the medical syringes is able to be selected by color.

Preferably, the colors of the plungers 30, of the several syringes 10 are associated with selected medications, families of medications, order of injection or other important factors in medical practice.

The enablements described in detail above are considered novel over the prior art of record and are considered critical to the operation of at least one aspect of one best mode embodiment of the instant invention and to the achievement of the above described objectives. The words used in this specification to describe the instant embodiments are to be understood not only in the sense of their commonly defined meanings, but to include by special definition in this specification: structure, material or acts beyond the scope of the commonly defined meanings. Thus if an element can be understood in the context of this specification as including more than one meaning, then its use must be understood as being generic to all possible meanings supported by the specification and by the word or words describing the element.

The definitions of the words or elements of the embodiments of the herein described invention and its related embodiments not described are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the invention and its various embodiments or that a single element may be substituted for two or more elements in a claim.

Changes from the claimed subject matter as viewed by a person with ordinary skill in the art, now known or later devised, are expressly contemplated as being equivalents within the scope of the invention and its various embodiments. Therefore, obvious substitutions now or later known to one with ordinary skill in the art are defined to be within the scope of the defined elements. The invention and its various embodiments are thus to be understood to include what is specifically illustrated and described above, what is conceptually equivalent, what can be obviously substituted, and also what essentially incorporates the essential idea of the invention.

While the invention has been described with reference to at least one preferred embodiment, it is to be clearly understood by those skilled in the art that the invention is not limited thereto. Rather, the scope of the invention is to be interpreted only in conjunction with the appended claims and it is made clear, here, that the inventor(s) believe that the claimed subject matter is the invention.

What is claimed is:

1. A medical syringe comprising: a barrel engaging a plunger and a cannula, the plunger movable in translation within the barrel, the cannula fixed at one end of the barrel and in fluid communication with the barrel; the barrel of a transparent material and supporting graduation indicia on a side wall of the barrel; the plunger of a non-transparent color contrasting with the graduation indicia whereby, the indicia is easily seen against the contrasting background of the plunger.

2. A plurality of medical syringes, each of the syringes comprising: a barrel engaging a plunger and a cannula, the plunger movable in translation within the barrel, the cannula fixed at one end of the barrel and in fluid communication with the barrel; the barrel of a transparent material and supporting graduation indicia on a side wall of the barrel; the plunger of a non-transparent color contrasting with the graduation indicia whereby, the indicia is easily seen against the contrasting background of the plunger; the plurality of medical syringes each having a unique plunger color whereby, each of the medical syringes is able to be selected by color.

3. The plurality of medical syringes of claim 2, wherein each of the colors of the plungers is associated with a selected medication.

4. The plurality of medical syringes of claim 2, wherein each of the colors of the plungers is associated with a selected syringe use.
